(19) United States
(12) Patent Application Publication
(10) Pub. No.: US 2018/0051330 A1
(43) Pub. Date: Feb. 22, 2018

(54) METHODS OF AMPLIFYING NUCLEIC ACIDS AND COMPOSITIONS AND KITS FOR PRACTICING THE SAME

(71) Applicant: Becton, Dickinson and Company, Franklin Lakes, NJ (US)

(72) Inventors: Terry J. Amiss, Raleigh, NC (US); Nicholas Herrmann, Holly Springs, NC (US); Richard Lee Kelley, Durham, NC (US); Frances Poyen Tong, Carrboro, NC (US); Eileen Snowden, Cary, NC (US)

(21) Appl. No.: 15/561,010

(22) PCT Filed: Mar. 29, 2016
(86) PCT No.: PCT/US2016/024739

§ 371 Date: Sep. 22, 2017

Related U.S. Application Data
(60) Provisional application No. 62/142,947, filed on Apr. 3, 2015.

(51) Int. Cl. C12Q 1/68; G01N 33/48

(52) U.S. Cl. CPC C12Q 1/6869; C12M 1/00; C12Q 2545/107; G01N 33/48

(57) ABSTRACT

Provided are methods of amplifying nucleic acids. The methods include combining a nucleic acid sample, a known amount of one or more competitive internal standard nucleic acids, and one or more amplification primers adapted to amplify one or more nucleic acids of interest present in the nucleic acid sample and the one or more competitive internal standard nucleic acids. The nucleic acid sample, competitive internal standard nucleic acids, and amplification primers are combined in a reaction mixture under conditions sufficient to amplify the one or more nucleic acids of interest and the one or more competitive internal standard nucleic acids. Aspects of the present disclosure further include compositions and kits that find use in practicing embodiments of the methods.

### CROSS-REFERENCE TO RELATED APPLICATIONS

Pursuant to 35 U.S.C. § 119(e), this application claims priority to the filing date of U.S. Provisional Patent Application No. 62/142,947, filed Apr. 3, 2015; the disclosure of which application is herein incorporated by reference.

### INTRODUCTION

Nucleic acid sequencing methods include the Sanger “dideoxy” method, which method relies upon the use of dideoxyribonucleoside triphosphates as chain terminators. The Sanger method has been adapted for use in automated sequencing with the use of chain terminators incorporating fluorescent labels. Other methods include “next-generation” sequencing methods, including those based on successive cycles of incorporation of fluorescently labeled nucleic acid analogues. In such “sequencing by synthesis” or “cycle sequencing” methods the identity of the added base is determined after each nucleotide addition by detecting the fluorescent label. Other next-generation sequencing methods include those based on the detection of hydrogen ions that are released during the polymerization of DNA. A microwell containing a template DNA strand to be sequenced is flooded with a single species of deoxyribonucleotide triphosphate (dNTP). If the introduced dNTP is complementary to the leading template nucleotide, it is incorporated into the growing complementary strand. This incorporation causes the release of a hydrogen ion that triggers an ISFET ion sensor, which indicates that a reaction has occurred. If homopolymer repeats are present in the template sequence, multiple dNTP molecules will be incorporated in a single cycle. This leads to a corresponding number of released hydrogens and a proportionally higher electronic signal.

### SUMMARY

Provided are methods of amplifying nucleic acids. The methods include combining a nucleic acid sample, a known amount of one or more competitive internal standard nucleic acids, and one or more amplification primers adapted to amplify one or more nucleic acids of interest present in the nucleic acid sample and the one or more competitive internal standard nucleic acids. The nucleic acid sample, competitive internal standard nucleic acids, and amplification primers are combined in a reaction mixture under conditions sufficient to amplify the one or more nucleic acids of interest and the one or more competitive internal standard nucleic acids. Aspects of the present disclosure further include compositions and kits that find use in practicing embodiments of the methods.

### BRIEF DESCRIPTION OF THE FIGURES

FIG. 1 shows a process for preparing nucleic acid samples for sequencing according to one embodiment of the present disclosure. Due to the complex nature of biological samples and multi-step process needed to ready a sample for sequencing, there can be significant variability in the coverage breadth and depth. To relate the number of reads obtained during sequencing for, e.g., any given microorganism, a tumor variant, etc., a competitive internal standard nucleic acid was used to correct for these variables.

FIG. 2 shows the sequence of a competitive internal standard nucleic acid according to one embodiment of the present disclosure. In this example, the competitive internal standard nucleic acid is a rpoB competitive internal standard nucleic acid. The primers are designated by the arrows, while the identifying mutation (introducing a restriction site) is indicated in yellow.

FIG. 3 shows sequencing read data obtained using a method according to one embodiment of the present disclosure. The read data was graphed versus the E. coli copy number (left) and the IS copy number (right).

FIG. 4 shows the calculation of read ratios according to one embodiment of the present disclosure using the read data shown in FIG. 3. The ratios were graphed (left) and then used to back-calculate E. coli copies and the expected versus calculated copies were graphed (right).

FIGS. 5A and 5B show the design and PCR amplification of three competitive internal standard nucleic acids according to one embodiment of the present disclosure. The design of the three competitive internal standard nucleic acids for the AmpliSeq™ cancer panel v2 is shown (FIG. 5A). The primer sequences are indicated with yellow and the identifying base pair changes are indicated using red letters. The variants identified in the TNBC samples are highlighted red. Shown on the right (FIG. 5B) is a PCR amplification of the competitive internal standard nucleic acids using either a single primer pair or the IT AmpliSeq™ primer pool containing 207 primer pairs. Both amplifications showed product of the correct size of approximately 200 bp.

FIG. 6 shows data from TNBC samples run without competitive internal standard nucleic acids sequenced with external controls using the AmpliSeq™ Cancer Hotspot Panel v2 and an Ion Torrent™ PGM sequencing system with a 316 chip. The graph shows allele frequency for variants called. For the 1.4 and 14 cell controls, mutations in MCF-7 gDNA and MCF-7 cultured cells occurring at high frequency (red) are highly correlated. In cultured cells, mutations not identified in gDNA were of low frequency. The gDNA for the HCT15 was a sequencing control and the variants identified agreed with previous analysis.

FIG. 7 shows data from samples run without competitive internal standard nucleic acids, which were examined for variant allele frequency, coverage depth, and amino acid translation. Only three mutations produced altered proteins: MET N375S, KRAS G12A and identified only in single cells TP53 P72R. TP53 is the most commonly mutated gene in human cancer and mutations at codon 72 have been studied extensively due to its association with cancer susceptibility and poor prognosis.

FIG. 8 shows data relating to read quality and depth for the competitive internal standard nucleic acids. Two controls are plotted “Blank IS-0.01” and “SC IS-0” and are a positive control (the competitive internal standard nucleic acids added to water) and a negative control (a single cell without the competitive internal standard nucleic acids) respectively. The negative control (x), as expected, did not have any competitive internal standard nucleic acid reads. While the positive control (blue diamonds) showed reads for MET and TP53, but not KRAS. Both SC samples amplified with competitive internal standard nucleic acids showed all of the competitive internal standard nucleic acid variants expected and the SC with the higher concentration of competitive internal standard nucleic acid demonstrated a higher number of reads.

FIG. 9 shows data relating variants identified for the TNBC samples NGS experiment with competitive internal standard nucleic acids added to some samples. At the top of the heat plot the samples are labeled IS-0.01 for awater blank with competitive internal standard nucleic acids added, then SC-0, SC-1, SC-0.01 for a single cell with no competitive internal standard nucleic acids or with 1 or 0.01 copies respectively of the competitive internal standard nucleic acids. The blue boxes are placed around the competitive internal standard nucleic acids base pair changes that are used to identify the competitive internal standard nucleic acids. The inset table gives the sequencing reads and ratios for the IS.

### DETAILED DESCRIPTION

Provided are methods of amplifying nucleic acids. The methods include combining a nucleic acid sample, a known amount of one or more competitive internal standard nucleic acids, and one or more amplification primers adapted to amplify one or more nucleic acids of interest present in the nucleic acid sample and the one or more competitive internal standard nucleic acids. The nucleic acid sample, competitive internal standard nucleic acids, and amplification primers are combined in a reaction mixture under conditions sufficient to amplify the one or more nucleic acids of interest and the one or more competitive internal standard nucleic acids. Aspects of the present disclosure further include compositions and kits that find use in practicing embodiments of the methods.

Before the methods, compositions and kits of the present disclosure are described in greater detail, it is to be understood that the methods, compositions and kits are not limited to particular embodiments described, as such may, of course, vary. It is also to be understood that the terminology used herein is for the purpose of describing particular embodiments only, and is not intended to be limiting, since the scope of the methods, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, compositions and kits. The upper and lower limits of these smaller ranges may independently be included in the smaller ranges and are also encompassed within the methods, compositions and kits, subject to any specifically excluded limit in the stated range. Where the stated range includes one or both of the limits, ranges excluding either or both of those included limits are also included in the methods, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, compositions and kits belong. Although any methods, compositions and kits similar or equivalent to those described herein can also be used in the practice or testing of the methods, compositions and kits, representative illustrative methods, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, compositions and kits, which are, for clarity, described in the context of separate embodiments, may also be provided in combination in a single embodiment. Conversely, various features of the methods, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions/kits. In addition, all sub-combinations listed in the embodiments describing such variables are also specifically embraced by the present methods, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods, compositions and kits. Any recited method can be carried out in the order of events recited or in any other order that is logically possible.

### Methods

Aspects of the present disclosure include methods of amplifying nucleic acids. The methods include combining a nucleic acid sample, a known amount of one or more competitive internal standard nucleic acids, and one or more amplification primers adapted to amplify one or more nucleic acids of interest present in the nucleic acid sample and the one or more competitive internal standard nucleic acids. The one or more competitive internal standard nucleic acids include a mismatch relative to one or more corresponding nucleic acids in the nucleic acid sample. The nucleic acid sample, competitive internal standard nucleic acids, and amplification primers are combined in a reaction mixture under conditions sufficient to amplify the one or more nucleic acids of interest and the one or more competitive internal standard nucleic acids.

Nucleic Acid Samples

The nucleic acid sample may be any nucleic acid sample that includes, or is suspected of including, one or more nucleic acids of interest, e.g., one or more nucleic acids for which amplification of the one or more nucleic acids is desirable. Amplification of the one or more nucleic acids may be desirable for a variety of reasons, including but not limited to, sequencing the amplification products (or “amplicons”) of the one or more nucleic acids of interest. Sequencing the amplification products enables one to determine the nucleotide sequence(s) of the one or more nucleic acids of interest and, optionally, to quantify the amount of the one or more nucleic acids of interest present in the nucleic acid sample.

The nucleic acid sample may be one or more cells, or a nucleic acid sample isolated from one or more cells. For example, the nucleic acid sample may be a nucleic acid sample isolated from a single cell, a plurality of cells (e.g., cultured cells), a tissue, an organ, or an organism (e.g., bacteria, yeast, or the like). In certain aspects, the nucleic acid sample is isolated from a cell(s), tissue, organ, and/or the like of a mammal (e.g., a human, a rodent (e.g., a mouse), or any other mammal of interest). In other aspects, the nucleic acid sample is isolated from a source other than a mammal, such as bacteria, yeast, insects (e.g., drosophila), amphibians (e.g., frogs (e.g., Xenopus)), viruses, plants, or any other non-mammalian nucleic acid sample source.

According to certain embodiments, the nucleic acid sample is isolated from a biological sample, such as a biological fluid or a biological tissue. Examples of biological fluids include urine, blood, plasma, serum, saliva, semen, stool, sputum, cerebral spinal fluid, tears, mucus, sperm, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cells.

In certain aspects, the nucleic acid sample is isolated from a microorganism. Microorganisms of interest include, e.g., bacteria, fungi, yeasts, protozoans, viruses (including both non-enveloped and enveloped viruses), bacterial endospores (for example, Bacillus (including Bacillus anthracis, Bacillus cereus, and Bacillus subtilis) and Clostridium (including Clostridium botulinum, Clostridium difficile, and Clostridium perfringens)), and combinations thereof. Genera of microorganisms of interest include, but are not limited to, Listeria, Escherichia, Salmonella, Campylobacter, Clostridium, Helicobacter, Mycobacterium, Staphylococcus, Shigella, Enterococcus, Bacillus, Neisseria, Shigella, Streptococcus, Vibrio, Yersinia, Bordetella, Borrelia, Pseudomonas, Saccharomyces, Candida, and the like, and combinations thereof. Specific microorganism strains of interest include, but are not limited to, Escherichia coli, Yersinia enterocolitica, Yersinia pseudotuberculosis, Vibrio cholerae, Vibrio parahaemolyticus, Vibrio vulnificus, Listeria monocytogenes, Staphylococcus aureus, Salmonella enterica, Saccharomyces cerevisiae, Candida albicans, Staphylococcal enterotoxin ssp, Bacillus cereus, Bacillus anthracis, Bacillus atrophaeus, Bacillus subtilis, Clostridium perfringens, Clostridium botulinum, Clostridium difficile, Enterobacter sakazakii, Pseudomonas aeruginosa, and the like, and combinations thereof (preferably, Staphylococcus aureus, Salmonella enterica, Saccharomyces cerevisiae, Bacillus atrophaeus, Bacillus subtilis, Escherichia coli, human-infecting non-enveloped enteric viruses for which Escherichia coli bacteriophage is a surrogate, and combinations thereof).

According to certain embodiments, the nucleic acid sample is a tumor nucleic acid sample (that is, a nucleic acid sample isolated from a tumor). “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like.

According to certain embodiments, the nucleic acid sample is a deoxyribonucleic acid (DNA) sample. DNA samples of interest include, but are not limited to, genomic DNA samples, mitochondrial DNA samples, complementary DNA (cDNA, synthesized from any RNA or DNA of interest) samples, recombinant DNA samples (e.g., plasmid DNA samples), and any other DNA samples of interest.

In certain aspects, the nucleic acid sample is a ribonucleic acid (RNA) sample. RNA samples of interest include, but are not limited to, messenger RNA (mRNA) samples, small/short interfering RNA (siRNA) samples, microRNA (miRNA) samples, any other DNA samples of interest.

Approaches, reagents and kits for isolating DNA and RNA from sources of interest are known in the art and commercially available. For example, kits for isolating DNA from a source of interest include the DNeasy®, RNeasy®, QIAamp®, QIAprep® and QIAquick® nucleic acid isolation/purification kits by Qiagen, Inc. (Germantown, Md.); the DNAzol®, ChargeSwitch®, Purelink®, GeneCatcher® nucleic acid isolation/purification kits by Life Technologies, Inc. (Carlsbad, Calif.); the NucleoMag®, NucleoSpin®, and NucleoBond® nucleic acid isolation/purification kits by Clontech Laboratories, Inc. (Mountain View, Calif.). In certain aspects, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md.), the RecoverAll® Total Nucleic Acid Isolation kit for FFPE by Life Technologies, Inc. (Carlsbad, Calif.), and the NucleoSpin® FFPE kits by Clontech Laboratories, Inc. (Mountain View, Calif.).

When it is desirable to control the size of the nucleic acids in the nucleic acid sample, the sample may be subjected to shearing/fragmentation, e.g., to generate nucleic acids that are shorter in length as compared to precursor non-sheared nucleic acids (e.g., genomic DNA) in the original sample. Suitable shearing/fragmentation strategies include, but are not limited to, passing the sample one or more times through a micropipette tip or fine-gauge needle, nebulizing the sample, sonicating the sample (e.g., using a focused-ultrasonicator by Covaris, Inc. (Woburn, Mass.)), bead-mediated shearing, enzymatic shearing (e.g., using one or more DNA-shearing e.g., restriction, enzymes), chemical based fragmentation, e.g., using divalent cations, fragmentation buffer (which may be used in combination with heat) or any other suitable approach for shearing/fragmenting precursor nucleic acids to generate a shorter nucleic acids. In certain aspects, the nucleic acids generated by shearing/fragmentation of a starting nucleic acid sample has a length of from 50 to 10,000 nucleotides, from 100 to 5000 nucleotides, from 150 to 2500 nucleotides, from 200 to 1000 nucleotides, e.g., from 250 to 500 nucleotides in length. According to certain embodiments, the nucleic acids generated by shearing/fragmentation of a starting nucleic acid sample has a length of from 10 to 20 nucleotides, from 20 to 30 nucleotides, from 30 to 40 nucleotides, from 40 to 50 nucleotides, from 50 to 60 nucleotides, from 60 to 70 nucleotides, from 70 to 80 nucleotides, from 80 to 90 nucleotides, from 90 to 100 nucleotides, from 100 to 150 nucleotides, from 150 to 200, from 200 to 250 nucleotides in length, or from 200 to 1000 nucleotides or even from 1000 to 10,000 nucleotides, for example, as appropriate for a sequencing platform in which one desires to sequence amplicons produced upon amplification of the one or more nucleic acids of interest and the one or more competitive internal standard nucleic acids.

Competitive Internal Standard Nucleic Acids

As summarized above, according to the nucleic acid amplification methods of the present disclosure, a known amount of one or more competitive internal standard nucleic acids is combined into the reaction mixture. The one or more competitive internal standard nucleic acids include a mismatch relative to one or more corresponding nucleic acids in the nucleic acid sample. As used herein, a “competitive internal standard nucleic acid” is a nucleic acid that is not present in (e.g., is exogenous to) the nucleic acid sample, but is amplifiable using a primer that is also suitable for amplifying a corresponding nucleic acid present in the nucleic acid sample. In this way, the competitive internal standard nucleic acid “competes” for primer binding with the corresponding nucleic acid present in the nucleic acid sample. Because the competitive internal standard nucleic acid includes a mismatch relative to the corresponding nucleic acid present in the nucleic acid sample, amplicons produced from the competitive internal standard nucleic acid are distinguishable from amplicons produced from the corresponding nucleic acid present in the nucleic acid sample (e.g., distinguishable upon sequencing the amplicons, digesting the amplicons using a restriction enzyme that recognizes a site created or destroyed by the mismatch, etc.).

In certain aspects, the one or more competitive internal standard nucleic acids are designed/selected by a practitioner of the subject methods based on the type of nucleic acid sample that will be present in the reaction mixture. For example, the one or more competitive internal standard nucleic acids may be designed/selected to ensure that the one or more competitive internal standard nucleic acids will have one or more corresponding nucleic acids in the nucleic acid sample with which to compete for primer binding. By way of example, if the nucleic acid sample is a human genomic DNA sample or a human RNA sample isolated from a particular cell type, the one or more competitive internal standard nucleic acids may be designed/selected to correspond to one or more nucleic acid regions present in human genomic DNA (e.g., an exonic region, an intronic region, an intergenic region, all or a portion of a gene (e.g., a single copy gene, a multiple copy gene, and the like), combinations thereof, etc.), or one or more RNAs transcribed in the particular cell type (or cDNAs derived therefrom), respectively. Also by way of example, if the nucleic acid sample is a DNA or RNA sample isolated from a microorganism (or a sample suspected of including a microorganism), the one or more competitive internal standard nucleic acids may be designed/selected to correspond to one or more nucleic acids present in that microorganism.

The nucleic acid sequences present in the genomes, transcriptomes, etc. of nucleic acid sources of interest (e.g., human cells, microorganisms, etc.) are readily available from resources such as the nucleic acid sequence databases of the National Center for Biotechnology Information (NCBI), the European Molecular Biology Laboratory-European Bioinformatics Institute (EMBL-EBI), and the like. Based on such sequence information, one can design/select one or more competitive internal standard nucleic acids suitable for a particular nucleic acid sample employed in the methods of the present disclosure.

As just one example, in certain aspects, the nucleic acid sample is a bacterial DNA sample, and the one or more competitive internal standard nucleic acids corresponds to (but has one or more mismatches relative to) all or a portion of a polymerase gene present in the nucleic acid sample. The polymerase gene may be a DNA polymerase gene. Alternatively, the polymerase gene may be an RNA polymerase gene. In certain aspects, the polymerase gene is an RNA polymerase gene, where the RNA polymerase gene encodes the beta subunit of RNA polymerase (rpoB). Genes such as rpoB are useful, e.g., due to their presence in the vast majority of microorganisms, as well as their discriminatory power and ability to segregate species. Bacterial genes such as rpoB, which has been used for phylogenetic analysis and identification of bacteria, are useful, e.g., when studying closely related isolates.

As a further example, according to certain embodiments, the nucleic acid sample may be a tumor nucleic acid sample, e.g., a nucleic acid sample isolated from one or more tumor cells, such as one or more rare tumor cells (e.g., one or more triple-negative breast cancer cells (TNBCs, which test negative for estrogen receptors (ER−), progesterone receptors (PR−), and HER2 (HER2−)). According to one embodiment, when the nucleic acid sample is a tumor nucleic acid sample, the one or more competitive internal standard nucleic acids includes a competitive internal standard nucleic acid selected from a competitive internal standard nucleic acid including a region from a KRAS gene, a competitive internal standard nucleic acid including a region from a MET gene, a competitive internal standard nucleic acid including a region from a TP53 gene, and any combination thereof. For example, when the nucleic acid sample is a tumor nucleic acid sample, the one or more competitive internal standard nucleic acids includes each of a competitive internal standard nucleic acid including a region from a KRAS gene, a competitive internal standard nucleic acid including a region from a MET gene, and a competitive internal standard nucleic acid including a region from a TP53 gene.

The one or more competitive internal standard nucleic acids may include any desired number of mismatches relative to the corresponding nucleic acid(s) in the nucleic acid sample. In certain aspects, a competitive internal standard nucleic acid of the one or more competitive internal standard nucleic acids includes from 1 to 100, from 1 to 90, from 1 to 80, from 1 to 70, from 1 to 60, from 1 to 50, from 1 to 40, from 1 to 30, from 1 to 20, from 1 to 10 (e.g., 1, 2, 3, 4, 5, 6, 7, 8, 9, 10), from 1 to 5 mismatches (e.g., from 2 to 5 mismatches) relative to the corresponding nucleic acid in the nucleic acid sample.

According to certain embodiments, a competitive internal standard nucleic acid of the one or more competitive internal standard nucleic acids includes 1 mismatch, or 2 or more mismatches, such as 3 or more mismatches, 4 or more mismatches, 5 or more mismatches, 6 or more mismatches, 7 or more mismatches, 8 or more mismatches, 9 or more mismatches, 10 or more mismatches, 15 or more mismatches, 20 or more mismatches, 25 or more mismatches, 30 or more mismatches, 40 or more mismatches, or 50 or more mismatches relative to the corresponding nucleic acid in the nucleic acid sample. In certain aspects, a competitive internal standard nucleic acid of the one or more competitive internal standard nucleic acids includes 50 or fewer mismatches, 40 or fewer mismatches, 30 or fewer mismatches, 25 or fewer mismatches, 20 or fewer mismatches, 15 or fewer mismatches, 10 or fewer mismatches, 9 or fewer mismatches, 8 or fewer mismatches, 7 or fewer mismatches, 6 or fewer mismatches, 5 or fewer mismatches, 4 or fewer mismatches, 3 or fewer mismatches, 2 mismatches, or 1 mismatch relative to the corresponding nucleic acid in the nucleic acid sample.

When two or more competitive internal standard nucleic acids are employed, the number of mismatches in the competitive internal standard nucleic acids is independent from one another. That is, the number of mismatches may be the same or different among any of the two or more competitive internal standard nucleic acids employed.

When a competitive internal standard nucleic acid of the one or more competitive internal standard nucleic acids includes 2 or more mismatches, the number of nucleotides between adjacent mismatches (that is, the “spacing” between adjacent mismatches) may be known/predetermined based on the design/selection of the competitive internal standard nucleic acid. In certain aspects, the number of nucleotides between adjacent mismatches of the 2 or more mismatches is independently from 1 to 20 nucleotides, including from 1 to 15 nucleotides, from 1 to 10 nucleotides, from 1 to 8 nucleotides (e.g., from 4 to 8 nucleotides, such as 6 nucleotides), 5 nucleotides, 4 nucleotides, 3 nucleotides, 2 nucleotides, or 1 nucleotide. According to some embodiments, the number of nucleotides between adjacent mismatches of the 2 or more mismatches is independently 100 or fewer nucleotides, 50 or fewer nucleotides, 40 or fewer nucleotides, 30 or fewer nucleotides, 20 or fewer nucleotides, 15 or fewer nucleotides, 10 or fewer nucleotides, 8 or fewer nucleotides, 6 or fewer nucleotides, 5 or fewer nucleotides, 4 or fewer nucleotides, 3 or fewer nucleotides, 2 nucleotides, or 1 nucleotide. In certain aspects, the number of nucleotides between adjacent mismatches of the 2 or more mismatches is independently 1 or more nucleotides, 2 or more nucleotides, 3 or more nucleotides, 4 or more nucleotides, 5 or more nucleotides, 6 or more nucleotides, 8 or more nucleotides, 10 or more nucleotides, 15 or more nucleotides, 20 or more nucleotides, 30 or more nucleotides, 40 or more nucleotides, 50 or more nucleotides, or 100 or more nucleotides.

According to certain embodiments, the mismatch in a competitive internal standard nucleic acid creates/provides a restriction enzyme recognition site in the competitive internal standard nucleic acid that is not present in the corresponding nucleic acid of the nucleic acid sample. Such a mismatch finds use, e.g., in enabling one to distinguish the competitive internal standard nucleic acid (or amplicon thereof) from the corresponding nucleic acid of the nucleic acid sample (or amplicon thereof) via digestion with the restriction enzyme that recognizes the site that is only present in the presence of the mismatch. Here, digestion using the relevant restriction enzyme will result in a cleavage event within the competitive internal standard nucleic acid that does not occur in the corresponding nucleic acid of the nucleic acid sample. The restriction site can be used for rapid method development prior to NGS. Competitive internal standards are added to samples and processed as usual. Then samples are amplified by PCR, the amplicons are digested with a restriction enzyme and a sized-based analysis is performed. This protocol enables quantitation of both the native and internal standard concentration in the samples.

In other embodiments, the mismatch in a competitive internal standard nucleic acid causes the absence of a restriction enzyme recognition site in the competitive internal standard nucleic acid that is present in the corresponding nucleic acid of the nucleic acid sample. Such a mismatch finds use, e.g., in enabling one to distinguish the competitive internal standard nucleic acid (or amplicon thereof) from the corresponding nucleic acid of the nucleic acid sample (or amplicon thereof) via digestion with the restriction enzyme that recognizes the site that is only present in the absence of the mismatch. Here, digestion using the relevant restriction enzyme will result in a cleavage event within the corresponding nucleic acid of the nucleic acid sample that does not occur in the competitive internal standard nucleic acid.

The one or more competitive internal standard nucleic acids may be any suitable length. In certain aspects, the one or more competitive internal standard nucleic acids are, independently, from 10 to 500 nucleotides in length, such as from 10 to 400 nucleotides in length, from 10 to 300 nucleotides in length, from 10 to 275 nucleotides in length, from 10 to 250 nucleotides in length, from 10 to 225 nucleotides in length, from 10 to 200 nucleotides in length, from 10 to 175 nucleotides in length, from 10 to 150 nucleotides in length, from 10 to 125 nucleotides in length, from 10 to 100 nucleotides in length, from 10 to 75 nucleotides in length, or from 10 to 50 nucleotides in length.

A known amount of one or more competitive internal standard nucleic acids are combined into the reaction mixture. The known amount may be based on the number of each of the one or more competitive internal standard nucleic acids combined into the reaction mixture, the final concentration of each of the one or more competitive internal standard nucleic acids upon assembly of the final reaction mixture, and/or the like. In certain aspects, each of the one or more competitive internal standard nucleic acids is added in an amount, independently, of genome copy number of the unknown sample. In some instances a negative control (blank) many be analyzed, in other instances a single cell (6-7 pictograms), 10 cells (60-70 pictograms), 100 cells (600-700 pictograms), or more are analyzed.

Amplification Primers

As summarized above, according to the nucleic acid amplification methods of the present disclosure, combined into the reaction mixture are one or more amplification primers adapted to amplify one or more nucleic acids of interest present in the nucleic acid sample and the one or more competitive internal standard nucleic acids.

Any amplification primer, or combination of two or more amplification primers, adapted to amplify the one or more nucleic acids of interest and the one or more competitive internal standard nucleic acids may be employed. In certain aspects, the one or more amplification primers are random primers, e.g., oligonucleotides of random sequence capable of amplifying a heterogeneous population of nucleic acids, some of which have a sequence that permits amplification of both the one or more nucleic acids of interest and the one or more competitive internal standard nucleic acids.

In other aspects, the one or more amplification primers are non-random primers. When the one or more amplification primers are non-random primers, the non-random primer(s) may be specifically designed/selected to amplify one or more predetermined nucleic acids of interest in the sample and the one or more competitive internal standard nucleic acids. For example, the one or more amplification primers may be designed/selected by a practitioner of the subject methods based both on the type of nucleic acid sample that will be present in the reaction mixture and the one or more competitive internal standard nucleic acids employed in the method. By way of example, when the nucleic acid sample is a human genomic DNA sample, the one or more amplification primers may be designed/selected by the practitioner to ensure that the one or more amplification primers are adapted to amplify one or more nucleic acid regions of interest present in human genomic DNA (e.g., an exonic region, an intronic region, an intergenic region, combinations thereof, etc.) and the one or more competitive internal standard nucleic acids employed in the method. Also by way of example, when the nucleic acid sample is a human RNA sample isolated from a particular cell type, the one or more amplification primers may be designed/selected by the practitioner to ensure that the one or more amplification primers are adapted to amplify one or more RNAs transcribed in the particular cell type (or cDNAs derived therefrom) and the one or more competitive internal standard nucleic acids employed in the method. As a further example, when the nucleic acid sample is a DNA or RNA sample isolated from a microorganism (or a sample suspected of including a microorganism), the one or more amplification primers may be designed/selected by the practitioner to ensure that the one or more amplification primers are adapted to amplify one or more nucleic acids of interest present in that microorganism and the one or more competitive internal standard nucleic acids employed in the method.

According to certain embodiments, a “pool” (or “panel”) of two or more amplification primers is employed. Such pools find use, e.g., when multiplexed amplification of multiple nucleic acids or nucleic acid regions of interest is desirable, e.g., for exome sequencing, targeted sequencing, SNP genotyping/variant detection by sequencing, aneuploidy analysis, genomic profiling, expression profiling, and/or the like. In certain aspects, a pool of two or more amplification primers are designed/selected to amplify two or more regions of interest present in genomic DNA (e.g., human genomic DNA). For example, the two or more regions of interest present in genomic DNA may correspond to “hot spot” regions that are frequently mutated in human cancer genes. Such primer pools may be specifically designed by one practicing the subject methods, or the practitioner may order one of the various commercially available primer pools, such as an Ion AmpliSeq™ Cancer Hotspot Panel available from Life Technologies, Inc. (Carlsbad, Calif.).

When the one or more amplification primers employed in the subject methods are non-random primers, a primer of the one or more amplification primers may be designed to be sufficiently complementary to a competitive internal standard nucleic acid and the nucleic acid of interest in the nucleic acid sample corresponding to the competitive internal standard nucleic acid, such that the primer specifically hybridizes to a region of the competitive internal standard nucleic acid or the corresponding region of the nucleic acid of interest under hybridization conditions.

The term “complementary” as used herein refers to a nucleotide sequence that base-pairs by non-covalent bonds to a region of the competitive internal standard nucleic acid or the corresponding region of the nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, the amplification primer may be perfectly (i.e., 100%) complementary to the competitive internal standard nucleic acid or the corresponding region of the nucleic acid of interest, or the primer and the competitive internal standard nucleic acid or the corresponding region of the nucleic acid of interest may share some degree of complementarity which is less than perfect (e.g., 70%, 75%, 85%, 90%, 95%, 99%). The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

As used herein, the term “hybridization conditions” means conditions in which a primer specifically hybridizes to a region of the competitive internal standard nucleic acid or the corresponding region of the nucleic acid of interest. Whether a primer specifically hybridizes to a target nucleic acid is determined by such factors as the degree of complementarity between the polymer and the target nucleic acid and the temperature at which the hybridization occurs, which may be informed by the melting temperature (T_{M}) of the primer. The melting temperature refers to the temperature at which half of the primer-target nucleic acid duplexes remain hybridized and half of the duplexes dissociate into single strands. The Tₘ of a duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3rd ed., Cold Spring Harbor Press, Cold Spring Harbor, N.Y., Ch. 10). Other more advanced models that depend on various parameters may also be used to predict Tₘ of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

In certain aspects, the one or more amplification primers include a sequencing adapter (e.g., 5′ relative to a 3′ hybridization region of the primer(s)). By “sequencing adapter” is meant one or more nucleic acid domains that include at least a portion of a nucleic acid sequence (or complement thereof) utilized by a sequencing platform of interest, such as a sequencing platform provided by Illumina® (e.g., the HiSeq™, MiSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems); or any other sequencing platform of interest.

In certain aspects, the one or more amplification primers include a sequencing adapter that includes a nucleic acid domain selected from: a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind); a barcode domain (e.g., a domain that uniquely identifies the sample source of the nucleic acid being sequenced to enable sample multiplexing by marking every molecule from a given sample with a specific barcode or “tag”); a barcode sequencing primer binding domain (a domain to which a primer used for sequencing a barcode binds); a molecular identification domain (e.g., a molecular index tag, such as a randomized tag of 4, 6, or other number of nucleotides) for uniquely marking molecules of interest to determine expression levels based on the number of instances a unique tag is sequenced; a complement of any such domains; or any combination thereof. In certain aspects, a barcode domain (e.g., sample index tag) and a molecular identification domain (e.g., a molecular index tag) may be included in the same nucleic acid.

The one or more amplification primers may include a sequencing adapter of any length and sequence suitable for the sequencing platform of interest. In certain aspects, the nucleic acid domains are from 4 to 100 nucleotides in length, such as from 6 to 75, from 8 to 50, or from 10 to 40 nucleotides in length.

The one or more amplification primers may include one or more nucleotides (or analogs thereof) that are modified or otherwise non-naturally occurring. For example, the amplification primers may include one or more nucleotide analogs (e.g., LNA, FANA, 2′-O-Me RNA, 2′-fluoro RNA, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to the primers and/or resulting amplicons.

Reaction Conditions

As summarized above, the nucleic acid sample, the known amount of one or more competitive internal standard nucleic acids, and the one or more amplification primers are combined in a reaction mixture under conditions sufficient to amplify the one or more nucleic acids of interest and the one or more competitive internal standard nucleic acids. By “conditions sufficient to amplify the one or more nucleic acids of interest and the one or more competitive internal standard nucleic acids” is meant reaction conditions that permit polymerase-mediated extension of a 3′ end of the one or more amplification primers. Achieving suitable reaction conditions may include selecting reaction mixture components, concentrations thereof, and a reaction temperature to create an environment in which a polymerase is active and the relevant nucleic acids in the reaction interact (e.g., hybridize) with one another in the desired manner. Suitable hybridization conditions are described in detail above.

In addition to the nucleic acid sample, the known amount of one or more competitive internal standard nucleic acids, the one or more amplification primers, a polymerase, and dNTPs, the reaction mixture may include buffer components that establish an appropriate pH, salt concentration (e.g., KCl concentration), metal cofactor concentration (e.g., Mg²⁺ or Mn²⁺ concentration), and the like, for the extension reaction to occur. Other components may be included, such as one or more nuclease inhibitors (e.g., a DNase inhibitor and/or an RNase inhibitor), one or more additives for facilitating amplification/replication of GC rich sequences, one or more enzyme-stabilizing components (e.g., DTT present at a final concentration ranging from 1 to 10 mM (e.g., 5 mM)), and/or any other reaction mixture components useful for facilitating polymerase-mediated extension reactions. In certain aspects, when the template nucleic acid is RNA, and when the extension reaction has proceeded for a desired amount of time, RNase H is added to hydrolyze any template RNAs that hybridized to the nascent cDNA strands.

The reaction mixture can have a pH suitable for the primer extension reaction and template-switching. In certain embodiments, the pH of the reaction mixture ranges from 5 to 9, such as from 7 to 9. In some instances, the reaction mixture includes a pH adjusting agent. pH adjusting agents of interest include, but are not limited to, sodium hydroxide, hydrochloric acid, phosphoric acid buffer solution, citric acid buffer solution, and the like. For example, the pH of the reaction mixture can be adjusted to the desired range by adding an appropriate amount of the pH adjusting agent.

The temperature range suitable for amplification of the one or more nucleic acids of interest and the one or more competitive internal standard nucleic acids may vary according to factors such as the particular polymerase employed, the melting temperatures of the one or more amplification primers employed, etc. According to certain embodiments, the reaction mixture conditions include bringing the reaction mixture to a temperature ranging from 4° C. to 80° C., such as from 16° C. to 75° C., e.g., from 37° C. to 72° C.

Example Additional Embodiments

The methods of the present disclosure may include one or more steps in addition to the combining step described above. For example, the methods may further include utilizing the amplified one or more nucleic acids of interest and the amplified one or more competitive internal standard nucleic acids in a downstream application/assay of interest. The amplified one or more nucleic acids of interest and the amplified one or more competitive internal standard nucleic acids may be utilized directly (optionally after a purification step), or may be modified prior to being utilized in a downstream application/assay of interest.

In certain aspects, it may be desirable to sequence the amplification products (e.g., using a Sanger sequencing system, a next generation sequencing (NGS) system, or the like), where the addition of one or more sequencing adapters to the amplification products is useful or necessary for sequencing on a particular sequencing system of interest. Accordingly, in certain aspects, the methods further include adding a sequencing adapter to the amplified one or more nucleic acids of interest and the amplified one or more competitive internal standard nucleic acids. Such a step may be performed whether or not the amplified one or more nucleic acids of interest and the amplified one or more competitive internal standard nucleic acids already include one or more sequencing adapters (e.g., by virtue of the one or more amplification primers including one or more sequencing adapters as described above). Sequencing adapters that may be added to the amplified one or more nucleic acids of interest and the amplified one or more competitive internal standard nucleic acids include, e.g., one or more capture domains, one or more sequencing primer binding domains, one or more barcode domains, one or more barcode sequencing primer binding domains, one or more molecular identification domains, a complement of any such domains, or any combination thereof. Further details regarding sequencing adapters are described hereinabove.

According to certain embodiments, the methods include subjecting the amplified one or more nucleic acids of interest and the amplified one or more competitive internal standard nucleic acids to restriction enzyme digestion conditions in which either the one or more competitive internal standard nucleic acids or the amplified one or more nucleic acids of interest are cleaved by a restriction enzyme present in the digestion reaction. As described above, a mismatch in a competitive internal standard nucleic acid may create/provide a restriction enzyme recognition site in the competitive internal standard nucleic acid that is not present in the corresponding nucleic acid of the nucleic acid sample. Alternatively, a mismatch in a competitive internal standard nucleic acid may result in the absence of a restriction enzyme recognition site in the competitive internal standard nucleic acid that is present in the corresponding nucleic acid of interest of the nucleic acid sample. In this way, the mismatch finds use, e.g., in enabling one to distinguish the amplified one or more nucleic acids of interest and the amplified one or more competitive internal standard nucleic acids based on whether the restriction enzyme digests the amplified one or more nucleic acids of interest or the amplified one or more competitive internal standard nucleic acids.

In certain aspects, the methods include adding a sequencing adapter to the amplified one or more nucleic acids of interest and the amplified one or more competitive internal standard nucleic acids, and subjecting the amplified one or more nucleic acids of interest and the amplified one or more competitive internal standard nucleic acids to restriction enzyme digestion conditions, in any order as desired.

According to certain embodiments, the methods include sequencing the amplified one or more nucleic acids of interest and the amplified one or more competitive internal standard nucleic acids. Such amplification products may be sequenced directly (optionally after a purification step), or may be modified prior to being sequenced. Modifications prior to sequencing include, but are not limited to, the addition of one or more sequencing adapters as described above, subjecting the amplicons to restriction enzyme digestion conditions as described above, and/or any other useful modifications for sequencing the amplicons on a sequencing platform of interest.

The sequencing may be carried out on any suitable sequencing platform, including a Sanger sequencing platform, a next generation sequencing (NGS) platform (e.g., using a next generation sequencing protocol), or the like. NGS sequencing platforms of interest include, but are not limited to, a sequencing platform provided by Illumina® (e.g., the HiSeq™, MiSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems); or any other sequencing platform of interest. Detailed protocols for preparing the amplicons for sequencing (e.g., by further amplification (e.g., solid-phase amplification), or the like), sequencing the amplicons, and analyzing the sequencing data are available from the manufacturer of the sequencing system of interest.

In certain aspects, the methods further include determining the amount of one or more of the one or more nucleic acids of interest in the nucleic acid sample. Such a determination may be based on, e.g., the number of sequencing reads corresponding to nucleic acids of interest in the nucleic acid sample, the number of sequencing reads corresponding to the one or more competitive internal standard nucleic acids, and the known amount of the one or more competitive internal standard nucleic acids.

According to some embodiments, determining the amount of one or more of the one or more nucleic acids of interest in the nucleic acid sample includes determining a ratio of the number of sequencing reads corresponding to the one or more competitive internal standard nucleic acids and the known amount of the one or more competitive internal standard nucleic acids.

The ratio of the number of sequencing reads corresponding to the one or more competitive internal standard nucleic acids and the known amount of the one or more competitive internal standard nucleic acids is useful for a variety of purposes. In certain aspects, this ratio is utilized to determine the amount of nucleic acids of interest in the nucleic acid sample. Such a determination may be based on, e.g., the number of sequencing reads corresponding to nucleic acids of interest in the nucleic acid sample, and the ratio.

According to some embodiments, the following formula is used to determine the amount (in this example, the number of copies) of a nucleic acid of interest present in a nucleic acid sample:

c_NA/c_IS=r_NA/r_IS (Formula I)

where c=the number of copies, r=the number of sequencing reads, NA=the nucleic acid of interest, and IS=the competitive internal standard nucleic acid.

### Utility

The methods of the present disclosure (as well as the compositions, nucleic acids sequencing systems and kits described below) find use in a variety of applications, including but not limited to, applications in which it is desirable to determine the nucleotide sequence and/or amount of nucleic acids of interest present in a nucleic acid sample. Applications of interest include, e.g., research applications, clinical applications (e.g., clinical diagnostic applications), etc., and the methods may be employed in such applications to assess whether one or more nucleic acids of interest are present in a nucleic acid sample, determine the nucleotide sequences of the one or more nucleic acids of interest, and/or quantify the amount of the one or more nucleic acids of interest present in the sample.

The methods of the present disclosure—which employ competitive internal standards nucleic acids—provide advantages over existing approaches in a number of respects. For example, in certain embodiments, the methods of the present disclosure are advantageous in the context of nucleic acid sequencing for reasons including, but not limited to, the provision of quality control (QC) metrics, e.g., for improved characterization of the analysis quality of next generation sequencing samples. Such metrics include correcting for variability (e.g. sample loss), permitting evaluation of amplification and/or sequencing fidelity, etc. For example, because a known amount of the one or more internal standard nucleic acids may be present in each of the samples that are amplified and subsequently sequenced, differences in the numbers of sequencing reads across samples may indicate sample loss during the workflow, e.g., it may be inferred that a sample that produces a relatively low number of sequencing reads experienced a degree of loss during the workflow. Also by way of example, because the one or more internal standard nucleic acids have a known sequence, sequencing reads corresponding to the one or more internal standard nucleic acids which include errors relative to the sequences of the one or more internal standard nucleic acids indicates an issue with the fidelity of amplification and/or the sequencing runs.

In certain aspects, the methods of the present disclosure are advantageous in that they decrease the costs associated with sequencing analysis, e.g., next generation sequencing analysis. For example, the inclusion of the one or more internal standard nucleic acids obviates the need for certain cost-increasing quality control aspects of existing sequencing approaches, such as the need for replicate samples, the need to rerun samples on the same and/or different sequencing platform, the need for external controls (e.g., the need to run well characterized genomic DNA, cell lines, etc. side-by-side), and the like.

### Compositions

Aspects of the present disclosure further include compositions. The compositions of the present disclosure find a variety of uses, including in certain aspects, practicing the methods of the present disclosure.

According to certain embodiments, provided is a composition that includes a nucleic acid sample, a known amount of one or more competitive internal standard nucleic acids, where the one or more competitive internal standard nucleic acids include a mismatch relative to one or more corresponding nucleic acids in the nucleic acid sample, and one or more amplification primers adapted to amplify one or more nucleic acids of interest present in the nucleic acid sample and the one or more competitive internal standard nucleic acids. The composition may include any nucleic acid sample of interest, any suitable competitive internal standard nucleic acid(s), and any suitable amplification primer(s), including any of the nucleic acid samples, competitive internal standard nucleic acids, and amplification primers described above in the section relating to the methods of the present disclosure.

Other components which may be present in the compositions of the present disclosure include, but are not limited to, a polymerase, dNTPs, a buffer component that establishes an appropriate pH, a salt (e.g., NaCl, KCl, or the like), a metal cofactor (e.g., Mg²⁺, Mn²⁺, or the like), a nuclease inhibitor (e.g., a DNase inhibitor and/or an RNase inhibitor), an additive for facilitating amplification/replication of GC rich sequences, an enzyme-stabilizing component (e.g., DTT), any other reaction mixture components (e.g., useful for facilitating polymerase-mediated extension reactions), and any combination thereof.

In certain aspects, a composition of the present disclosure includes the amplicons produced by the methods of the present disclosure. According to certain embodiments, such compositions include the amplicons in purified form (e.g., substantially or completely separated from the amplification reaction mixture components). The amplicons may include a sequencing adapter provided during or after the amplification reaction as described above, and/or a subset of the amplicons (e.g., the amplified one or more competitive internal standard nucleic acids or the amplified one or more corresponding nucleic acids of interest) may be restriction enzyme digestion products.

Any of the compositions of the present disclosure may be present in a container. Suitable containers include, but are not limited to, tubes, vials, plates (e.g., a 96- or other-well plate).

Any of the compositions of the present disclosure may be present in a device. Devices of interest include, but are not limited to, an incubator, a thermocycler, a sequencing system (e.g., a Sanger sequencing system or a next generation sequencing system), a microfluidic device, or the like.

### Nucleic Acid Sequencing Systems

Also provided by the present disclosure are nucleic acid sequencing systems. According to certain embodiments, the nucleic acid sequencing systems find use in sequencing amplicons generated using the methods of the present disclosure.

In certain aspects, a sequencing system of the present disclosure includes a collection of nucleic acids. The collection of nucleic acids include amplicons corresponding to nucleic acids of interest present in a nucleic acid sample, and amplicons corresponding to a known amount of one or more competitive internal standard nucleic acids. The one or more competitive internal standard nucleic acids include a mismatch relative to one or more corresponding nucleic acids in the nucleic acid sample.

According to certain embodiments, the sequencing system includes amplicons generated from any of the one or more competitive internal standard nucleic acids and any of the nucleic acids of interest described above in the section relating to the methods of the present disclosure.

The amplicons may include a sequencing adapter provided during the amplification reaction that produced the amplicons (e.g., provided according to embodiments of the subject methods) and/or after the amplification reaction (e.g., provided according to embodiments of the subject methods). A subset of the amplicons (e.g., the amplified one or more competitive internal standard nucleic acids or the amplified one or more corresponding nucleic acids of interest) may be restriction enzyme digestion products, e.g., produced according to embodiments of the subject methods.

The sequencing system may be any sequencing system of interest, including a Sanger sequencing system, a next generation sequencing (NGS) system, or the like. In certain aspects the sequencing system is an NGS system. NGS systems of interest include, but are not limited to, a sequencing system provided by Illumina® (e.g., the HiSeq™, MiSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems), or any other suitable NGS systems.

The collection of nucleic acids may be present in a component of the sequencing system. By way of example, the collection of nucleic acids may be present in a sample preparation component of the sequencing system, e.g., a component of the sequencing system where nucleic acids of the collection are fragmented and/or sequencing adapters are added to the nucleic acids of the collection. Also by way of example, the collection of nucleic acids may be present in a solid-phase amplification component of the sequencing system, where solid-phase amplification of the nucleic acids of the collection may occur. An example of such a solid-phase amplification component of a sequencing system is the flow cell of Illumina-based sequencing systems, where cluster generation occurs. Another example of such a solid-phase amplification component of a sequencing system is the Ion OneTouch™ 2 component for producing templates suitable for sequencing on an Ion PGM™ system, Ion Proton™ system, or other NGS system provided by Ion Torrent™. The collection of nucleic acids may be present in any component of a sequencing system useful for utilizing the collection of nucleic acids to obtain the nucleic acid sequences thereof.

According to certain embodiments, the sequencing system is adapted to determine the amount of nucleic acids of interest in the nucleic acid sample. In certain aspects, the determination is based on the number of sequencing reads corresponding to nucleic acids of interest in the nucleic acid sample, the number of sequencing reads corresponding to the one or more competitive internal standard nucleic acids, and the known amount of the one or more competitive internal standard nucleic acids. In certain aspects, such a sequencing system is adapted to determine a ratio of the number of sequencing reads corresponding to the one or more competitive internal standard nucleic acids to the known amount of the one or more competitive internal standard nucleic acids. When the sequencing system is adapted to determine such a ratio, the system may be further adapted to determine the amount of nucleic acids of interest in the nucleic acid sample based on the number of sequencing reads corresponding to nucleic acids of interest in the nucleic acid sample, and the ratio of the number of sequencing reads corresponding to the one or more competitive internal standard nucleic acids and the known amount of the one or more competitive internal standard nucleic acids.

By “adapted to determine the amount of nucleic acids of interest in the nucleic acid sample,” “adapted to determine a ratio of the number of sequencing reads corresponding to the one or more competitive internal standard nucleic acids to the known amount of the one or more competitive internal standard nucleic acids,” and the like, is meant that the sequencing system includes the components and functionality to perform the recited determinations. For example, in certain aspects, the sequencing system includes a processor and a computer-readable medium (e.g., a non-transitory computer-readable medium). The computer-readable medium includes instructions executable by the processor to, e.g., determine the amount of nucleic acids of interest in the nucleic acid sample as described above, determine a ratio of the number of sequencing reads corresponding to the one or more competitive internal standard nucleic acids to the known amount of the one or more competitive internal standard nucleic acids as described above, and/or the like.

### Kits

As summarize above, the present disclosure provides kits. According to certain embodiments, the kits include one or more competitive internal standard nucleic acids comprise a mismatch relative to one or more corresponding nucleic acids present in a nucleic acid sample of interest, and a container (e.g., a tube). In certain aspects, the one or more competitive internal standard nucleic acids are present in the container.

The subject kits may include any competitive internal standard nucleic acid(s) useful in a particular application of interest, and may include any of the one or more competitive internal standard nucleic acids described above in relation to the methods of the present disclosure.

Any other components or reagents useful, e.g., in practicing the methods of the present disclosure, may be included in the subject kits. In certain aspects, the kits further include one or more amplification primers for amplifying the one or more competitive internal standard nucleic acids and one or more nucleic acids of interest present in a sample of interest. According to certain embodiments, the kits include one or more of a polymerase, dNTPs, a buffer component that establishes an appropriate pH, a salt (e.g., NaCl, KCl, or the like), a metal cofactor (e.g., Mg²⁺, Mn²⁺, or the like), a nuclease inhibitor (e.g., a DNase inhibitor and/or an RNase inhibitor), an additive for facilitating amplification/replication of GC rich sequences, an enzyme-stabilizing component (e.g., DTT), and/or any other reaction mixture components, e.g., useful for facilitating polymerase-mediated extension reactions.

In certain aspects, a kit of the present disclosure further includes one or more reagents for performing a restriction enzyme digestion reaction, e.g., for digesting amplicons produced from the one or more competitive internal standard nucleic acids or amplicons produced from one or more nucleic acids of interest present in a sample of interest.

Components of the subject kits may be present in separate containers, or multiple components may be present in a single container. For example, when two or more competitive internal nucleic acids are included in the kit, each of the two or more competitive internal standard nucleic acids may be present in separate containers, subsets of the two or more competitive internal standard nucleic acids may be present in separate containers, each of the two or more competitive internal standard nucleic acids may be present in a single container, etc.

The one or more competitive internal standard nucleic acids may be provided in any suitable container. For example, the population may be provided in a single tube (e.g., vial), in one or more wells of a plate (e.g., a 96-well plate, a 384-well plate, etc.), or the like.

In addition to the above-mentioned components, a kit of the present disclosure may further include instructions for using the components of the kit, e.g., to practice the methods of the present disclosure. For example, the kit may include instructions for using the one or more competitive internal standard nucleic acids to determine the amount of one or more genes of interest present in a nucleic acid sample of interest. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1

### Next-Generation Sequencing (NGS) Analysis of Microorganisms

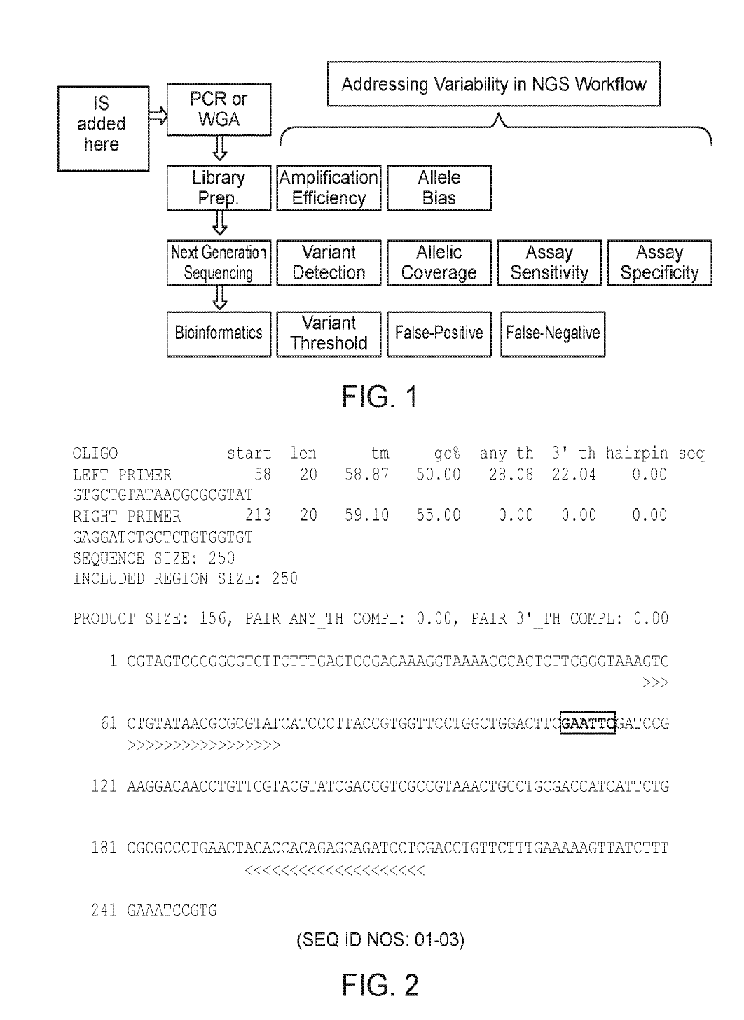

In the present example and Example 2 below, at the initiation of sample processing, internal standards (IS) are added to samples at known concentrations to correct for variability and sample loss during the NGS workflow (FIG. 1). This is accomplished by relating both the number of IS reads to the IS starting concentration and incorporating this ratio into calculations for concentrations of unknowns.

For the NGS analysis of microorganisms, employed in this example was an internal standard (IS) containing a region of the rpoB gene. This gene was chosen due to its presence in the vast majority of microorganisms and its discriminatory power and ability to segregate species. rpoB encodes the beta-subunit of RNA polymerase and is used for phylogenetic analysis and identification of bacteria, especially when studying closely related isolates. To differentiate the IS from native rpoB sequences, unique and identifiable mutations were designed and placed in synthetic DNA. The rpoB IS has two base pair modifications that create a restriction site which can be used prior to sequencing to differentiate it from native sequences. FIG. 2 gives the 200 bp IS sequence and primers used for its amplification. The 200 bp IS were synthesized and ligated into a plasmid manufactured at Life Technologies using GeneArt® Gene Synthesis.

To test the IS, the purified plasmid containing rpoB was added, at known concentrations, to standard concentrations of E. coli gDNA prior to NGS sample preparation. The samples were then amplified using the rpoB primers in a standard reaction mix. To sequence the amplicons, they were first purified, end repaired, repurified, then ligated to Ion Torrent adaptors and repurified again. Prior to clonal amplification the DNA libraries were quantified using the TapeStation system (Agilent Technologies). Clonal amplification was performed on the Ion OneTouch™ system, then samples were loaded onto a 316 chip and sequenced according to manufacturer instructions. The data was processed using the Ion Torrent™ Browser and aligned an average of 94% of the sample DNA to the rpoB gene.

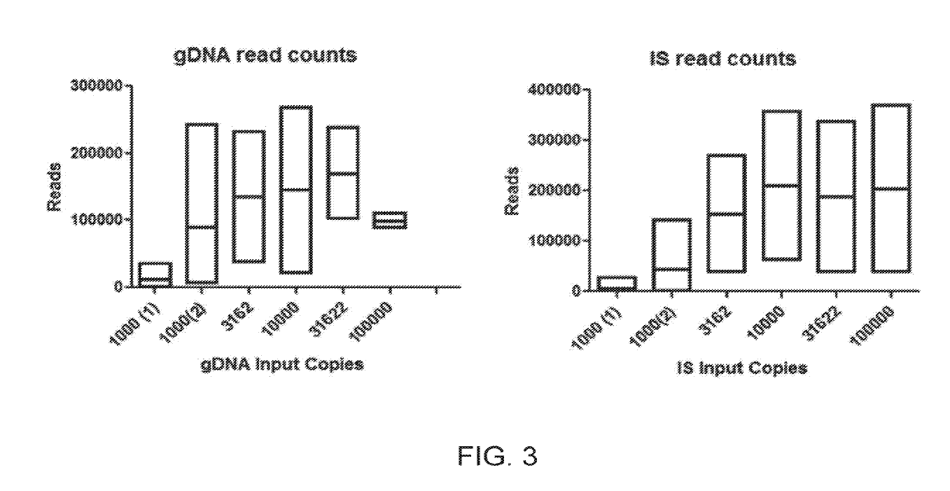

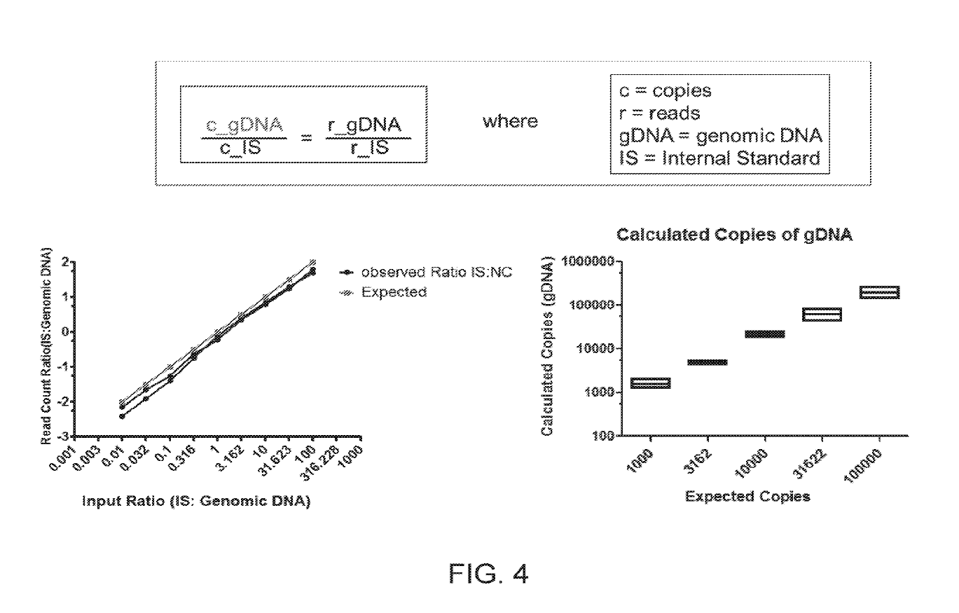

The mean read length ranged from 131 to 135 bp and had a mean coverage depth of 11,302 at AQ20. The reads versus DNA concentration of the native and IS rpoB amplicons was graphed (FIG. 3). In general, the number of reads increased as the sample gDNA concentration increased, however as seen in the bar chart the variability was high. To demonstrate that the IS corrects for this variability, a new plot was generated using the IS to calculate ratios, according to the formula (FIG. 4). Graphing the ratio greatly diminished the variability (R2=0.98) and this graph was used for E. coli quantitation which was calculated to be within 2-fold of the actual copy number.

### Example 2

### Next-Generation Sequencing (NGS) Analysis of Tumor Samples

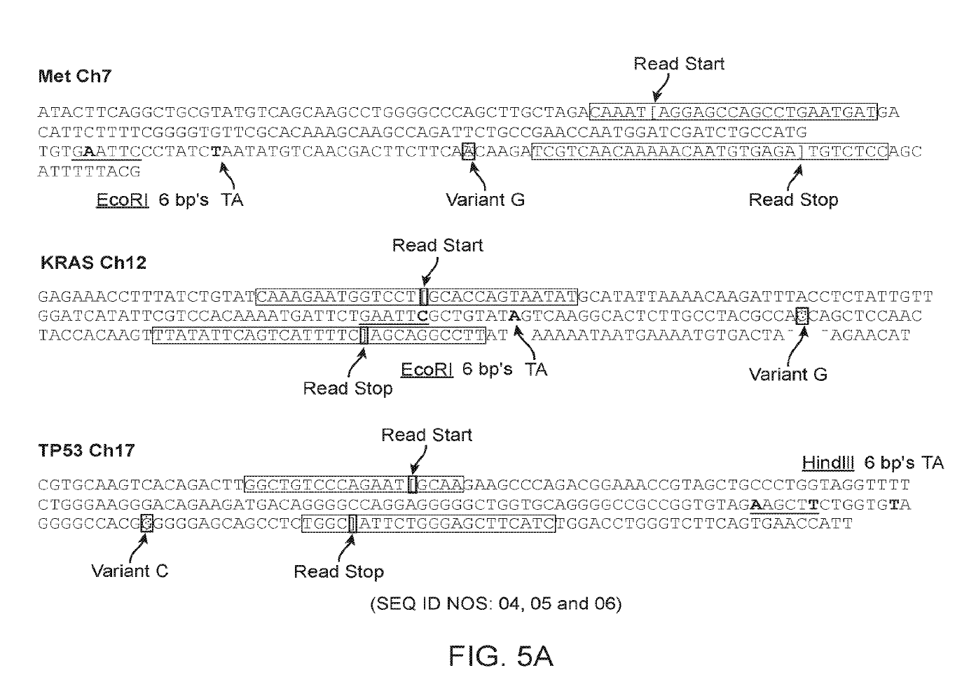

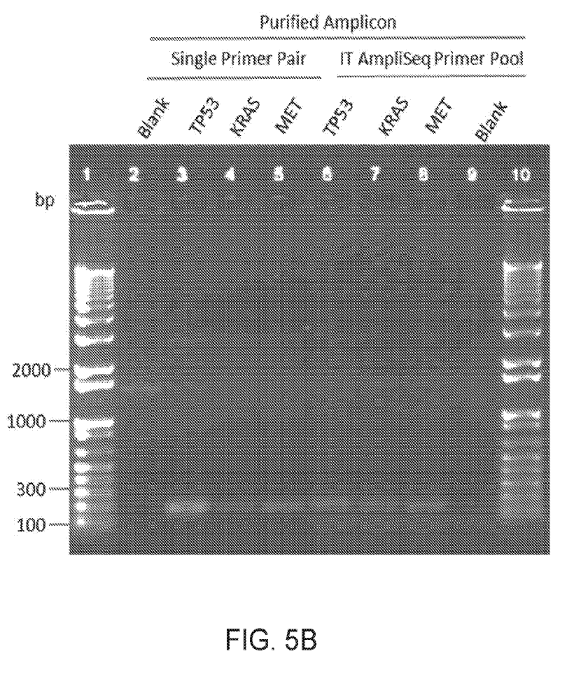

For analysis of tumor samples, an AmpliSeq™ cancer panel was run with and without internal standards (IS). AmpliSeq targeted sequencing was performed on clinical tumor specimens grown in a patient-derived xenograft (PDX) mouse. The tumor was classified as a spindle cell metaplastic carcinoma ER, PR negative and Her2-neu negative (TNBC). The TNBC samples were used to develop methods to sequence rare cells. For this reason the TNBC samples were flow sorted in aliquots containing single, ten or fifty cells. Due to the small amount of DNA in the samples, whole genome amplification (Repli-g) was used to obtain the quantities of DNA needed for sequencing. The first sequencing run on the TNBC cells was done using well-characterized cell lines run side-by-side as controls. While the second sequencing run used an IS spiked into the sample and no external controls. For the IS, three plasmids containing the KRAS, MET and TP53 sequences were designed to have unique base pair changes enabling their identification (FIGS. 5A & 5B). MET and KRAS IS have two identifying base pair changes, while the TP53 has three. These changes add a restriction site, then 6 nucleotides downstream, there are either one or two base pair changes. Depending on the experiment the internal standard can be added as a plasmid or alternatively, may be added as a linear fragment of DNA containing the internal standard KRAS, MET or TP53 nucleic acid sequences.

Experiment without IS

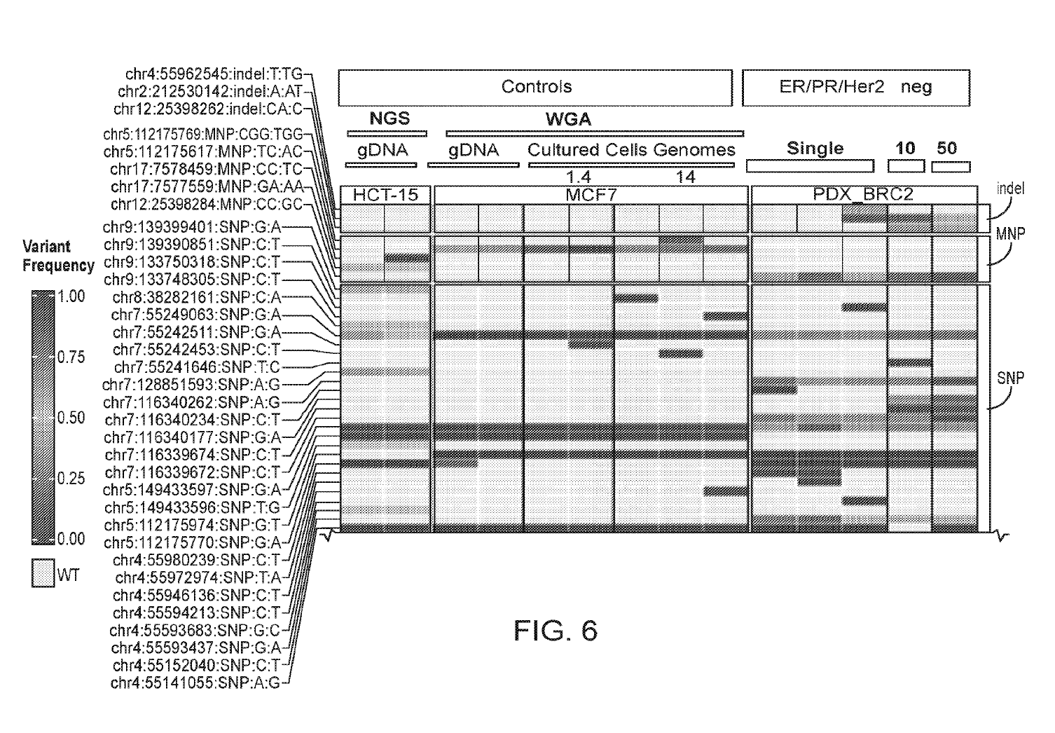

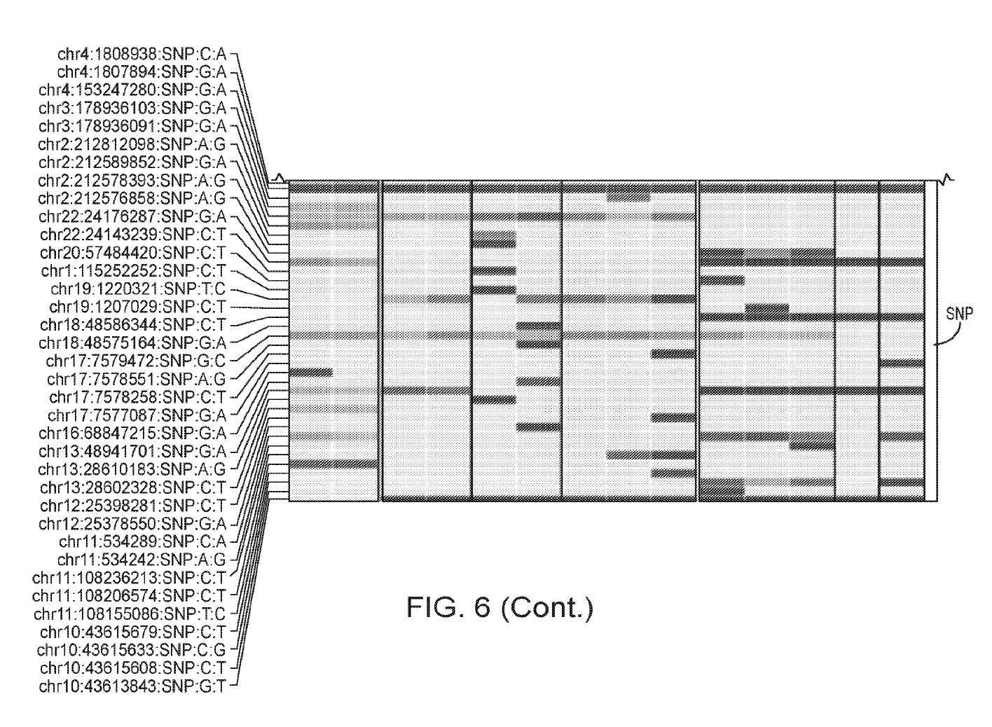

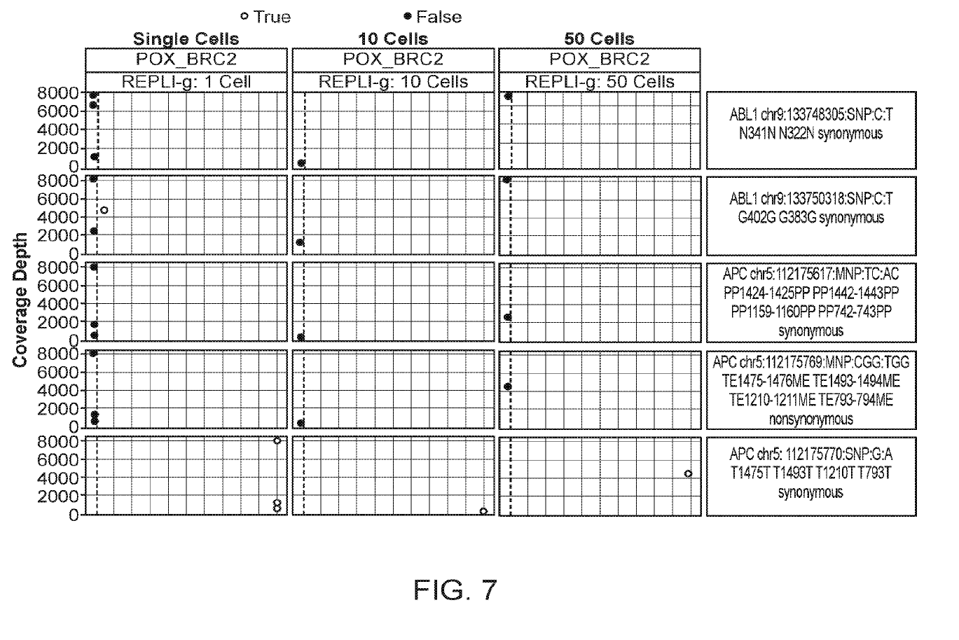

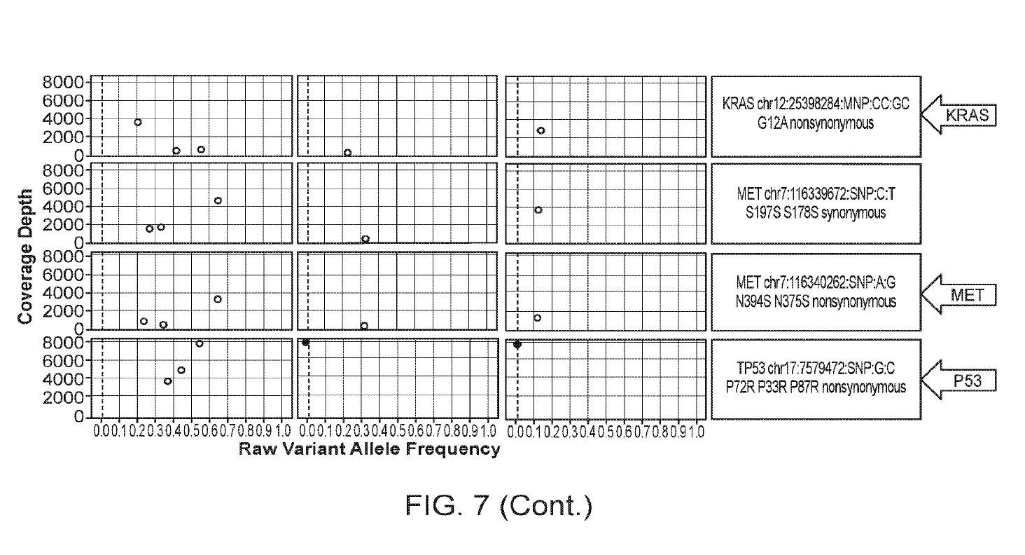

TNBC tissue from the PDX mouse was index sorted using the FACSAria™ II flow cytometry system. The sorting was done using a cocktail of two anti-mouse reagents: CD45 and H2Kd to ensure that only human cells were selected. Samples of single, ten or fifty cells were sorted directly into a PCR 96 well plate. For external controls, HCT15 and MCF7 gDNA and cultured MCF7 cells were run side-by-side the TNBC samples. The MCF7 cells were grown in standard media, washed and diluted in PBS to the desired number of cells. Quantitation of these cells was accomplished using the Kapa Bio hgDNA quantitation kit. Prior to sample processing for AmpliSeq™ sequencing, the TNBC and MCF7 cells and the MCF7 gDNA were amplified using Repli-g according to standard protocols. After amplification the DNA was purified and AmpliSeq™ sequencing libraries were produced. The variants and their frequencies were graphed for the five TNBC and the eight control samples (FIG. 6). The three sequenced single cells each had 22-23 mutations, most of these were silent. Although, three variants were identified that affect their gene product. These three mutations were in the KRAS, MET and TP53 genes (FIG. 7).

Experiment with IS

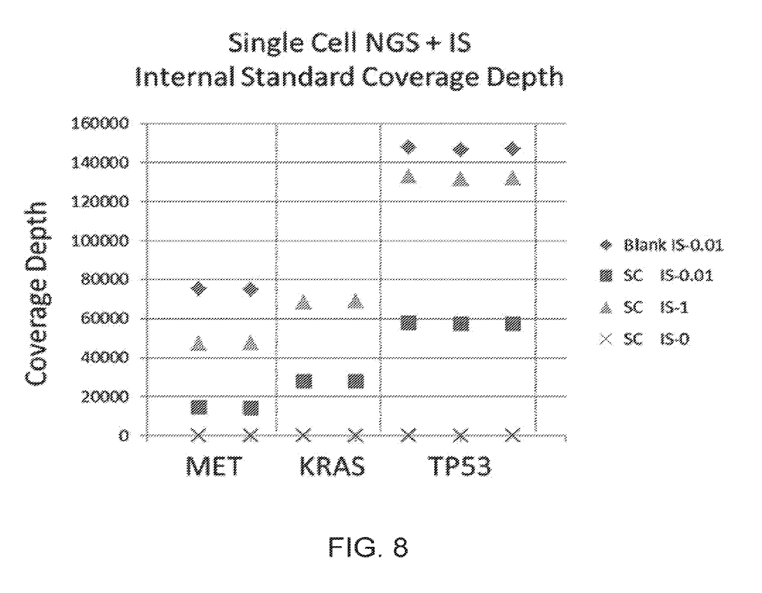

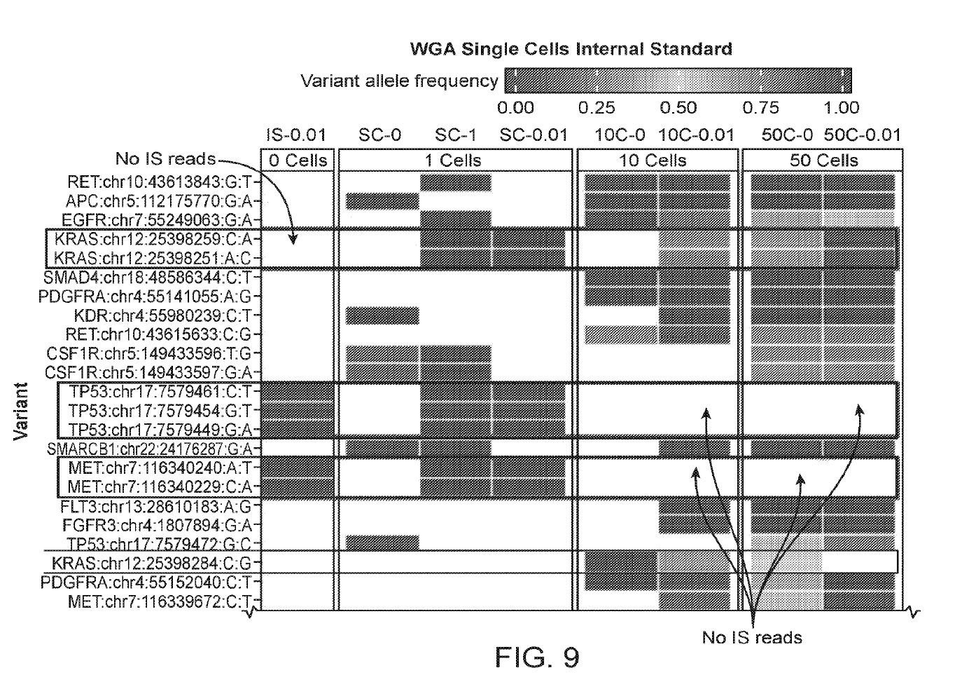

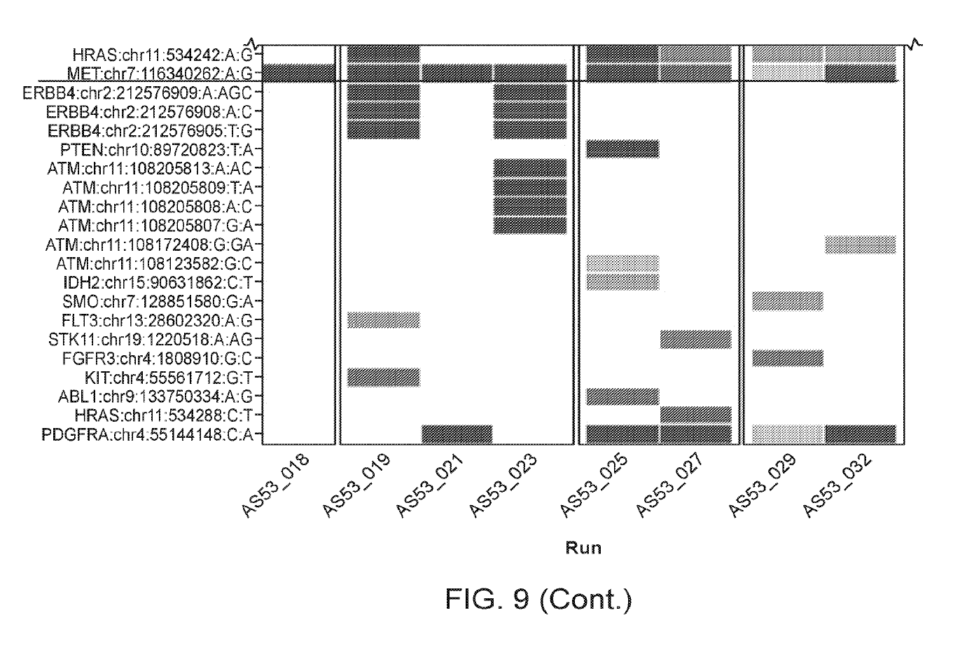

A mixture (1:1:1 ratio) of the three IS plasmids containing KRAS, MET and TP53 sequences were added to TNBC cells (single, ten or fifty cells) prior to Repli-g. One of two concentrations of IS was added to the samples. The higher concentration was calculated to be equal to 1 copy of the amplicon and the lower concentration was 100-fold less (0.01 copy). Then, AmpliSeq™ NGS was performed and the samples were evaluated for IS reads and read quality. In FIG. 8, high quality reads from the Ion Torrent variant tables were plotted for each engineered mutation in the IS. The negative control (x), as expected, did not have any IS reads. While the positive control (blue diamonds) showed reads for MET and TP53, but not KRAS. This may indicate that KRAS was affected by primer annealing bias and/or amplification bias since the NGS reads were absent. When IS was spiked into single cells, reads for each of the IS was present indicating that KRAS, MET and TP53 IS sequences can be successfully processed and sequenced. In FIG. 9 the data was graphed with the relative variant allele frequency and the ratio of IS reads (inset Table). The ratio of the IS reads demonstrate variability due to bias and processing differences which are used mathematically to correct for bias in sample reads.

Notwithstanding the appended clauses, the disclosure set forth herein is also defined by the following clauses:
1. A method of amplifying nucleic acids, comprising:
   combining:
      a nucleic acid sample;
      a known amount of one or more competitive internal standard nucleic acids, wherein the one or more competitive internal standard nucleic acids comprise a mismatch relative to one or more corresponding nucleic acids in the nucleic acid sample; and
      one or more amplification primers adapted to amplify one or more nucleic acids of interest present in the nucleic acid sample and the one or more competitive internal standard nucleic acids,
   in a reaction mixture under conditions sufficient to amplify the one or more nucleic acids of interest and the one or more competitive internal standard nucleic acids.
2. The method according to Clause 1, wherein the one or more competitive internal standard nucleic acids comprises from 1 to 5 mismatches relative to one or more corresponding nucleic acids in the nucleic acid sample.
3. The method according to Clause 1, wherein the one or more competitive internal standard nucleic acids comprises from 2 or more mismatches relative to one or more corresponding nucleic acids in the nucleic acid sample.
4. The method according to Clause 3, wherein the 2 or more mismatches comprise a known number of nucleotides therebetween.
5. The method according to Clause 4, wherein the known number of nucleotides between adjacent mismatches of the 2 or more mismatches is independently from 2 to 20 nucleotides.
6. The method according to Clause 4, wherein the known number of nucleotides between adjacent mismatches of the 2 or more mismatches is independently from 4 to 8 nucleotides.
7. The method according to any one of Clauses 1 to 6, wherein the one or more competitive internal standard nucleic acids comprises a mismatch relative to one or more corresponding nucleic acids in the nucleic acid sample that creates a restriction enzyme recognition site in the one or more competitive internal standard nucleic acids that is not present in the one or more corresponding nucleic acids in the nucleic acid sample.
8. The method according to any one of Clauses 1 to 7, wherein the nucleic acid sample comprises nucleic acids isolated from one or more cells of a cellular sample of interest.
9. The method according to Clause 8, wherein the cellular sample of interest is a single cell.
10. The method according to any one of Clauses 1 to 9, wherein the nucleic acid sample comprises genomic DNA from a genome of interest.
11. The method according to Clause 10, wherein at least one of the one or more competitive internal standard nucleic acids corresponds to a single copy gene present in the genome of interest.
12. The method according to any one of Clauses 1 to 11, wherein the nucleic acid sample is a microorganism nucleic acid sample.
13. The method according to any one of Clauses 1 to 12, wherein the microorganism is a bacterium.
14. The method according to Clause 13, wherein the one or more competitive internal standard nucleic acids comprises a region of a polymerase gene.
15. The method according to Clause 14, wherein the polymerase gene is an RNA polymerase gene.
16. The method according to Clause 15, wherein the RNA polymerase gene encodes the beta subunit of RNA polymerase (rpoB).
17. The method according to any one of Clauses 1 to 11, wherein the nucleic acid sample is a tumor nucleic acid sample.
18. The method according to Clause 17, wherein the one or more competitive internal standard nucleic acids comprises a competitive internal standard nucleic acid selected from the group consisting of: a competitive internal standard nucleic acid comprising a region from a KRAS gene, a competitive internal standard nucleic acid comprising a region from a MET gene, a competitive internal standard nucleic acid comprising a region from a TP53 gene, and combinations thereof.
19. The method according to Clause 18, wherein the one or more competitive internal standard nucleic acids comprises each of a competitive internal standard nucleic acid comprising a region from a KRAS gene, a competitive internal standard nucleic acid comprising a region from a MET gene, and a competitive internal standard nucleic acid comprising a region from a TP53 gene.
20. The method according to any one of Clauses 1 to 19, wherein the one or more amplification primers comprise a sequencing adapter.
21. The method according to any one of Clauses 1 to 20, wherein the one or more amplification primers are non-random primers.
22. The method according to any one of Clauses 1 to 21, further comprising adding a sequencing adapter to the amplified one or more nucleic acids of interest and the amplified one or more competitive internal standard nucleic acids.
23. The method according to any one of Clauses 1 to 22, further comprising sequencing the amplified one or more nucleic acids of interest and the amplified one or more competitive internal standard nucleic acids.
24. The method according to Clause 23, wherein the sequencing is by a next generation sequencing protocol.
25. The method according to Clause 23 or Clause 24, further comprising determining the amount of nucleic acids of interest in the nucleic acid sample based on:
   the number of sequencing reads corresponding to nucleic acids of interest in the nucleic acid sample;
   the number of sequencing reads corresponding to the one or more competitive internal standard nucleic acids; and
   the known amount of the one or more competitive internal standard nucleic acids.
26. The method according to Clause 25, wherein determining the amount of nucleic acids of interest in the nucleic acid sample comprises determining a ratio of the number of sequencing reads corresponding to the one or more competitive internal standard nucleic acids and the known amount of the one or more competitive internal standard nucleic acids.
27. The method according to Clause 26, wherein the determining the amount of nucleic acids of interest in the nucleic acid sample is based on:
   the number of sequencing reads corresponding to nucleic acids of interest in the nucleic acid sample; and
   the ratio of the number of sequencing reads corresponding to the one or more competitive internal standard nucleic acids and the known amount of the one or more competitive internal standard nucleic acids.

28. A composition, comprising:
a nucleic acid sample;
a known amount of one or more competitive internal standard nucleic acids, wherein the one or more competitive internal standard nucleic acids comprise a mismatch relative to one or more corresponding nucleic acids in the nucleic acid sample; and
one or more amplification primers adapted to amplify one or more nucleic acids of interest present in the nucleic acid sample and the one or more competitive internal standard nucleic acids.
29. The composition according to Clause 28, wherein the one or more competitive internal standard nucleic acids comprises from 1 to 5 mismatches relative to one or more corresponding nucleic acids in the nucleic acid sample.
30. The composition according to Clause 28, wherein the one or more competitive internal standard nucleic acids comprises from 2 to 5 mismatches relative to one or more corresponding nucleic acids in the nucleic acid sample.
31. The composition according to Clause 30, wherein the from 2 to 5 mismatches comprise a known number of nucleotides therebetween.
32. The composition according to Clause 31, wherein the known number of nucleotides between the from 2 to 5 mismatches is independently from 2 to 20 nucleotides.
33. The composition according to Clause 31, wherein the known number of nucleotides between the from 2 to 5 mismatches is independently from 4 to 8 nucleotides.
34. The composition according to any one of Clauses 28 to 33, wherein the one or more competitive internal standard nucleic acids comprises a mismatch relative to one or more corresponding nucleic acids in the nucleic acid sample that creates a restriction enzyme recognition site in the one or more competitive internal standard nucleic acids that is not present in the one or more corresponding nucleic acids in the nucleic acid sample.
35. The composition according to any one of Clauses 28 to 34, wherein the nucleic acid sample comprises nucleic acids isolated from one or more cells of a cellular sample of interest.
36. The composition according to Clause 35, wherein the cellular sample of interest is a single cell.
37. The composition according to any one of Clauses 28 to 36, wherein the nucleic acid sample comprises genomic DNA from a genome of interest.
38. The composition according to Clause 37, wherein at least one of the one or more competitive internal standard nucleic acids corresponds to a single copy gene present in the genome of interest.
39. The composition according to any one of Clauses 28 to 38, wherein the nucleic acid sample is a microorganism nucleic acid sample.
40. The composition according to Clause 39, wherein the microorganism is a bacterium.
41. The composition according to Clause 40, wherein the one or more competitive internal standard nucleic acids comprises a region of a polymerase gene.
42. The composition according to Clause 41, wherein the polymerase gene is an RNA polymerase gene.
43. The composition according to Clause 42, wherein the RNA polymerase gene encodes the beta subunit of RNA polymerase (rpoB).
44. The composition according to any one of Clauses 28 to 38, wherein the nucleic acid sample is a tumor nucleic acid sample.
45. The composition according to Clause 44, wherein the one or more competitive internal standard nucleic acids comprises a competitive internal standard nucleic acid selected from the group consisting of: a competitive internal standard nucleic acid comprising a region from a KRAS gene, a competitive internal standard nucleic acid comprising a region from a MET gene, a competitive internal standard nucleic acid comprising a region from a TP53 gene, and combinations thereof.
46. The composition according to Clause 45, wherein the one or more competitive internal standard nucleic acids comprises each of a competitive internal standard nucleic acid comprising a region from a KRAS gene, a competitive internal standard nucleic acid comprising a region from a MET gene, and a competitive internal standard nucleic acid comprising a region from a TP53 gene.
47. The composition according to any one of Clauses 28 to 46, wherein the one or more amplification primers comprise a sequencing adapter.
48. The composition according to any one of Clauses 28 to 47, wherein the one or more amplification primers are not random primers.
49. A nucleic acid sequencing system, comprising:
a collection of nucleic acids comprising:
amplicons corresponding to nucleic acids of interest present in a nucleic acid sample; and
amplicons corresponding to a known amount of one or more competitive internal standard nucleic acids, wherein the one or more competitive internal standard nucleic acids comprise a mismatch relative to one or more corresponding nucleic acids in the nucleic acid sample.
50. The sequencing system according to Clause 49, wherein the one or more competitive internal standard nucleic acids comprises from 1 to 5 mismatches relative to one or more corresponding nucleic acids in the nucleic acid sample.
51. The sequencing system according to Clause 49, wherein the one or more competitive internal standard nucleic acids comprises from 2 or more mismatches relative to one or more corresponding nucleic acids in the nucleic acid sample.
52. The sequencing system according to Clause 51, wherein the 2 or more mismatches comprise a known number of nucleotides therebetween.
53. The sequencing system according to Clause 52, wherein the known number of nucleotides between adjacent mismatches of the 2 or more mismatches is independently from 2 to 20 nucleotides.
54. The sequencing system according to Clause 52, wherein the known number of nucleotides between adjacent mismatches of the 2 or more mismatches is independently from 4 to 8 nucleotides.
55. The sequencing system according to any one of Clauses 49 to 54, wherein the one or more competitive internal standard nucleic acids comprises a mismatch relative to one or more corresponding nucleic acids in the nucleic acid sample that creates a restriction enzyme recognition site in the one or more competitive internal standard nucleic acids that is not present in the one or more corresponding nucleic acids in the nucleic acid sample.
56. The sequencing system according to any one of Clauses 49 to 55, wherein the nucleic acid sample comprises nucleic acids isolated from one or more cells of a cellular sample of interest.
57. The sequencing system according to Clause 56, wherein the cellular sample of interest is a single cell.
58. The sequencing system according to any one of Clauses 49 to 57, wherein the nucleic acid sample comprises genomic DNA from a genome of interest.
59. The sequencing system according to Clause 58, wherein at least one of the one or more competitive internal standard nucleic acids corresponds to a single copy gene present in the genome of interest.
60. The sequencing system according to any one of Clauses 49 to 59, wherein the nucleic acid sample is a microorganism nucleic acid sample.
61. The sequencing system according to Clause 60, wherein the microorganism is a bacterium.
62. The sequencing system according to Clause 61, wherein the one or more competitive internal standard nucleic acids comprises a region of a polymerase gene.
63. The sequencing system according to Clause 62, wherein the polymerase gene is an RNA polymerase gene.
64. The sequencing system according to Clause 63, wherein the RNA polymerase gene encodes the beta subunit of RNA polymerase (rpoB).
65. The sequencing system according to any one of Clauses 49 to 59, wherein the nucleic acid sample is a tumor nucleic acid sample.
66. The sequencing system according to Clause 65, wherein the one or more competitive internal standard nucleic acids comprises a competitive internal standard nucleic acid selected from the group consisting of: a competitive internal standard nucleic acid comprising a region from a KRAS gene, a competitive internal standard nucleic acid comprising a region from a MET gene, a competitive internal standard nucleic acid comprising a region from a TP53 gene, and combinations thereof.
67. The sequencing system according to Clause 66, wherein the one or more competitive internal standard nucleic acids comprises each of a competitive internal standard nucleic acid comprising a region from a KRAS gene, a competitive internal standard nucleic acid comprising a region from a MET gene, and a competitive internal standard nucleic acid comprising a region from a TP53 gene.
68. The sequencing system according to any one of Clauses 49 to 67, wherein the amplicons were amplified using non-random primers.
69. The sequencing system according to any one of Clauses 49 to 68, wherein the sequencing system is adapted to determine the amount of nucleic acids of interest in the nucleic acid sample based on:
the number of sequencing reads corresponding to nucleic acids of interest in the nucleic acid sample;
the number of sequencing reads corresponding to the one or more competitive internal standard nucleic acids; and
the known amount of the one or more competitive internal standard nucleic acids.
70. The sequencing system according to Clause 69, wherein the sequencing system is adapted to determine a ratio of the number of sequencing reads corresponding to the one or more competitive internal standard nucleic acids to the known amount of the one or more competitive internal standard nucleic acids.
71. The sequencing system according to Clause 70, wherein the sequencing system is adapted to determine the amount of nucleic acids of interest in the nucleic acid sample based on:
the number of sequencing reads corresponding to nucleic acids of interest in the nucleic acid sample; and
the ratio of the number of sequencing reads corresponding to the one or more competitive internal standard nucleic acids and the known amount of the one or more competitive internal standard nucleic acids.
72. The sequencing system according to any one of Clauses 49 to 71, wherein the sequencing system is a next generation sequencing system.
73. A kit, comprising:
one or more competitive internal standard nucleic acids comprise a mismatch relative to one or more corresponding nucleic acids present in a nucleic acid sample of interest; and
a tube.
74. The kit according to Clause 73, wherein the one or more competitive internal standard nucleic acids comprises from 1 to 5 mismatches relative to one or more corresponding nucleic acids in the nucleic acid sample of interest.
75. The kit according to Clause 73, wherein the one or more competitive internal standard nucleic acids comprises from 2 or more mismatches relative to one or more corresponding nucleic acids in the nucleic acid sample.
76. The kit according to Clause 75, wherein the 2 or more mismatches comprise a known number of nucleotides therebetween.
77. The kit according to Clause 76, wherein the known number of nucleotides between adjacent mismatches of the 2 or more mismatches is independently from 2 to 20 nucleotides.
78. The kit according to Clause 76, wherein the known number of nucleotides between adjacent mismatches of the 2 or more mismatches is independently from 4 to 8 nucleotides.
79. The kit according to any one of Clauses 73 to 78, wherein the one or more competitive internal standard nucleic acids comprises a mismatch relative to one or more corresponding nucleic acids in the nucleic acid sample that creates a restriction enzyme recognition site in the one or more competitive internal standard nucleic acids that is not present in the one or more corresponding nucleic acids in the nucleic acid sample.
80. The kit according to any one of Clauses 73 to 79, wherein the nucleic acid sample comprises genomic DNA from a genome of interest.
81. The kit according to Clause 80, wherein at least one of the one or more competitive internal standard nucleic acids corresponds to a single copy gene present in the genome of interest.
82. The kit according to any one of Clauses 73 to 81, wherein the nucleic acid sample of interest is a microorganism nucleic acid sample.
83. The kit according to Clause 82, wherein the microorganism is a bacterium.
84. The kit according to Clause 83, wherein the one or more competitive internal standard nucleic acids comprises a region of a polymerase gene.
85. The kit according to Clause 84, wherein the polymerase gene is an RNA polymerase gene.
86. The kit according to Clause 85, wherein the RNA polymerase gene encodes the beta subunit of RNA polymerase (rpoB).
87. The kit according to any one of Clauses 73 to 81, wherein the nucleic acid sample is a tumor nucleic acid sample.
88. The kit according to Clause 87, wherein the one or more competitive internal standard nucleic acids comprises a competitive internal standard nucleic acid selected from the group consisting of: a competitive internal standard nucleic acid comprising a region from a KRAS gene, a competitive internal standard nucleic acid comprising a region from a MET gene, a competitive internal standard nucleic acid comprising a region from a TP53 gene, and combinations thereof.
89. The kit according to Clause 88, wherein the one or more competitive internal standard nucleic acids comprises each of a competitive internal standard nucleic acid comprising a region from a KRAS gene, a competitive internal standard nucleic acid comprising a region from a MET gene, and a competitive internal standard nucleic acid comprising a region from a TP53 gene.
90. The kit according to any one of Clauses 73 to 89, further comprising amplification primers adapted to amplify the one or more competitive internal standard nucleic acids.
91. The kit according to Clause 90, wherein the amplification primers comprise a sequencing adapter.
92. The kit according to any one of Clauses 90 to 91, wherein the amplification primers are non-random primers.
93. The kit according to any one of Clauses 73 to 92, further comprising instructions for using the one or more competitive internal standard nucleic acids to determine the amount of one or more genes of interest present in the nucleic acid sample of interest.
Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.
Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of amplifying nucleic acids, comprising:
combining:
a nucleic acid sample;
a known amount of one or more competitive internal standard nucleic acids, wherein the one or more competitive internal standard nucleic acids comprise a mismatch relative to one or more corresponding nucleic acids in the nucleic acid sample; and
one or more amplification primers adapted to amplify one or more nucleic acids of interest present in the nucleic acid sample and the one or more competitive internal standard nucleic acids,
in a reaction mixture under conditions sufficient to amplify the one or more nucleic acids of interest and the one or more competitive internal standard nucleic acids; and
sequencing the amplified one or more nucleic acids of interest and the amplified one or more competitive internal standard nucleic acids.

2. The method according to claim 1, wherein the one or more competitive internal standard nucleic acids comprises from 1 to 5 mismatches relative to one or more corresponding nucleic acids in the nucleic acid sample.

3. The method according to claim 1, wherein the one or more competitive internal standard nucleic acids comprises from 2 or more mismatches relative to one or more corresponding nucleic acids in the nucleic acid sample.

4. The method according to claim 3, wherein the 2 or more mismatches comprise a known number of nucleotides therebetween.

5. The method according to claim 4, wherein the known number of nucleotides between adjacent mismatches of the 2 or more mismatches is independently from 2 to 20 nucleotides.

6. The method according to claim 4, wherein the known number of nucleotides between adjacent mismatches of the 2 or more mismatches is independently from 4 to 8 nucleotides.

7. The method according to claim 1, wherein the one or more competitive internal standard nucleic acids comprises a mismatch relative to one or more corresponding nucleic acids in the nucleic acid sample that creates a restriction enzyme recognition site in the one or more competitive internal standard nucleic acids that is not present in the one or more corresponding nucleic acids in the nucleic acid sample.

8. The method according to claim 1, wherein the nucleic acid sample comprises nucleic acids isolated from one or more cells of a cellular sample of interest.

9. The method according to claim 8, wherein the cellular sample of interest is a single cell.

10. The method according to claim 1, wherein the nucleic acid sample comprises genomic DNA from a genome of interest.

11. The method according to claim 10, wherein at least one of the one or more competitive internal standard nucleic acids corresponds to a single copy gene present in the genome of interest.

12. A composition, comprising:
a nucleic acid sample;
a known amount of one or more competitive internal standard nucleic acids, wherein the one or more competitive internal standard nucleic acids comprise a mismatch relative to one or more corresponding nucleic acids in the nucleic acid sample; and
one or more amplification primers adapted to amplify one or more nucleic acids of interest present in the nucleic acid sample and the one or more competitive internal standard nucleic acids.

13. A nucleic acid sequencing system, comprising:
a collection of nucleic acids comprising:
amplicons corresponding to nucleic acids of interest present in a nucleic acid sample; and
amplicons corresponding to a known amount of one or more competitive internal standard nucleic acids, wherein the one or more competitive internal standard nucleic acids comprise a mismatch relative to one or more corresponding nucleic acids in the nucleic acid sample.

14. (canceled)
